(19) United States
(12) Patent Application Publication
(10) Pub. No.: US 2012/0157913 A1
(43) Pub. Date: Jun. 21, 2012

(54) CATHETER APPARATUS AND METHOD FOR ATHEROLYSIS

(75) Inventors: Kusai S. Aziz, Visalia, CA (US); Ross Tsugita, Mountain View, CA (US)

(73) Assignee: Atherolysis Medical, Inc., Visalia, CA (US)

(21) Appl. No.: 13/323,516

(22) Filed: Dec. 12, 2011

Related U.S. Application Data
(60) Provisional application No. 61/423,595, filed on Dec. 16, 2010.

(51) Int. Cl. A61M 5/168; A61M 1/00

(52) U.S. Cl. 604/28; 604/35; 604/31

(57) ABSTRACT

An atherolysis catheter comprises a catheter body and one or more isolation balloons. A distal section of the body comprises infusion and aspiration ports in fluid communication with infusion and aspiration lumens for delivering and collecting fluids. A pump delivers infusate through the infusion lumen, and a valve in the infusion port controls release of the infusate into a target location in the vasculature.

### CROSS-REFERENCES TO RELATED APPLICATIONS

The present application claims the benefit of provisional application No. 61/423,595 (attorney docket number 40463-703.101), filed on Dec. 16, 2010, the full disclosure of which is incorporated herein by reference.

### BACKGROUND OF THE INVENTION

1. Field of the Invention

The present application relates generally to medical devices and methods. More particularly, the invention herein relates to an atherolysis catheter apparatus for accessing, dissolving and/or modifying plaques formed in the vasculature of a patient.

Vascular diseases are the most common cause of morbidity and mortality in the United States. The major pathology is, for example, atherosclerosis, where plaque composed of lipids, calcium, and connective tissue builds up in the patient's vascular system and leads to blockages of the vascular system. These blockages typically occur in coronary arteries of the heart leading to angina or myocardial infarctions also known as heart attacks, in carotid arteries of the brain leading to brain ischemia and strokes, in renal arteries of the kidneys leading to renal artery stenosis and severe hypertension, and in arteries of the lower or upper extremities also referred to as peripheral vascular disease leading to pain in the limbs, difficulty in walking and gangrene. Plaque also accumulates in the aorta leading to atherosclerosis of the aorta and aortic aneurysms.

Conventional methods of managing vascular diseases caused by the buildup of plaque include changing the physical nature of the blockages, for example, by balloon angioplasty, by stenting in which the plaques are displaced to the artery's side walls, by atherectomy in which the plaque is cut and removed, and by bypass surgery in which a graft conduit is used to bypass the blockages, etc.

None of these conventional methods is effective in all cases, and there is a long felt but unresolved need for a method and an atherolysis catheter apparatus that can dissolve, modify or remove plaque accumulated in a particular region or a cavity of a patient's anatomy without interrupting blood supply to organs, for example, heart, brain, kidneys, extremities, etc., of the patient.

2. Description of the Background Art

U.S. Pat. No. 6,929,633 describes a thrombolytic infusion catheter with spaced-apart balloons and infusion and aspiration ports located between the balloons. U.S. Patent Publication No. 2005/0085769 describes a catheter having lumen configurations which achieve fluid exchange. U.S. Patent Publications 2010/0286589 and 2011/0196383, both of which are incorporated herein by reference and which have common ownership and inventorship with the present application, describe atherolytic compositions suitable for delivery by the methods and systems of the present invention.

### BRIEF SUMMARY OF THE INVENTION

The atherolysis catheter apparatus disclosed herein addresses the above stated need for an apparatus that can dissolve, modify, and/or remove plaque accumulated in a particular region, blood vessel (artery or vein), or a cavity of a patient's anatomy without interrupting blood supply to organs, for example, heart, brain, kidneys, extremities, etc., of the patient.

The present invention comprises an atherolysis catheter and methods of its use for delivering infusates to the vasculature for dissolving plaque, thrombus, and other occlusive materials associated with cardiovascular disease. While the occlusive materials will frequently be present in the arterial system, including both the coronary and the peripheral arterial systems, the occlusive materials may also be present in the venous vasculature, particularly in the peripheral venous vasculature where it may be associated with deep vein thrombosis and similar conditions.

Atherolysis catheters constructed in accordance with the principles of the present invention comprise a catheter body including at least one infusion lumen having at least one infusion port at a distal end thereof and an aspiration lumen having at least one aspiration port at a distal end thereof. Typically, the catheter body will include additional lumens and passages, such as a guide wire lumen, one or more balloon inflation lumens (for the optional isolation balloons which are discussed below), and one or more perfusion lumens to allow bypass blood flow during a therapeutic treatment, in particular when isolation balloons have been inflated which would otherwise block normal blood flow.

The atherolysis catheters of the present invention will also include a pump disposed in the infusion lumen, where the pump is adapted to induce flow of the infusate through the infusion lumen toward the infusion port. The atherolysis catheters will further include a pressure-responsive valve disposed at the infusion port, where the valve is normally closed (to block reflux of infusate blood, etc., through the infusion port) that opens in response to flow and pressure of the infusate caused by the pump.

In specific embodiments, the pressure-responsive valve may comprise a valve plug mounted in the infusion port, where the infusion port defines a valve seat against which the valve plug will rest when the opening pressure against the valve (i.e., the infusate pressure in the infusate lumen) is below a threshold level. The opening pressure threshold will be well above normal systolic levels to make sure that the valve opens regardless of patient blood pressure, typically being in the range from 10 psi to 100 psi, usually from 15 psi to 75 psi. The opening or “pop” pressure of the valve may be precisely adjusted using a spring which is attached to a lower end of the valve plug, where the spring is further attached to the catheter body, usually at a wall location in the infusion lumen opposite to the location of the infusion port.

The atherolysis catheters may have one, two, three, or more pressure-responsive valves, where the pressure-responsive valves may be adapted to open at the same pressure threshold or at different pressure thresholds. The valve plugs will usually have a conical taper which centers the plug in the infusion port when the plug seats and the valve is closed. When such a conically tapered valve plug opens, an annular gap is formed between the conical surface of the plug and the circular rim of the infusion port. This annular gap is particularly effective in acting as a nozzle jet to distribute the infusate laterally outward in a ring or conical pattern in the blood vessel. By properly controlling the pump, e.g., by cycling or pulsing the pump, the infusate may be released in a cyclic or pulsing pattern which helps mix the infusate with the plaque or thrombus material being treated in the blood vessel, thus promoting dissolution of the clot or thrombus.

In preferred embodiments of the present invention, the atherolysis catheter will further comprise a pair of axially spaced-apart isolation balloons, with a distal balloon disposed on the catheter body distally of the infusion port and aspiration port and a proximal balloon disposed on the catheter body proximally of the infusion port and the aspiration port. When such isolation balloons are incorporated into the atherolysis catheter, it will be preferred to include the perfusion lumen having an inlet port on one side of the pair of isolation balloons and an outlet port on the other side of the pair of isolation balloons.

The pump will include a rotor, impeller, or other active element disposed within the infusion lumen, preferably spaced closely to the infusion port(s) by a short distance, typically in the range from 5 cm to 25 cm. The rotor or impeller of the pump may be a conventional screw-type or turbine impeller, and will typically be driven by a separate motor. In some embodiments, the motor may be a small electric motor which itself is disposed in the infusion lumen, typically close to the pump impeller or rotor. Alternatively, the pump impeller or rotor may be driven by a drive cable or shaft which extends the length of the infusion lumen and which is driven by a motor which is located external to the infusion lumen and catheter, optionally being positioned in a proximal catheter hub. Usually, the drive motor for the pump will be adapted to be driven with a variable pattern, optionally in an on-off mode or a variable speed mode, where the motor slows and speeds up in a predetermined pattern. In both cases, the flow of infusate into the vasculature will be pulsed or variable in order to enhance mixing of the infusate as described above.

The present invention also provides methods for treating vascular occlusions. The methods comprise positioning a distal end of a catheter near an occlusion in a blood vessel, typically an artery but alternatively a vein in some cases. A lytic agent is pumped through an infusion lumen of the catheter and out an infusion port through a pressure-responsive valve. The valve is adapted to open at a particular threshold (as discussed above). Pressure and the geometry of the valve, typically a tapered plug, act to spray the lytic agent into the occlusive material in the blood vessel. The lytic agent and lysed products from the blood vessel are concurrently or successively aspirated through an aspiration port and aspiration lumen in the catheter body, typically by applying an external vacuum to a lumen but optionally by providing a second pump and motor in the aspiration lumen to draw the material outwardly.

The methods will usually employ a pump which is disposed within the infusion lumen, where the a pump maybe a rotating screw pump or other turbine or rotary pump. The screw pump may be driven by a motor which itself is present in the infusion lumen or by a motor which is external from the catheter. In the latter case, a drive cable may be disposed in the infusion lumen and used to couple the drive motor outside of the infusion lumen to the pump within the infusion lumen. Usually, distal and proximal isolation balloons will be inflated on either side of the infusion and aspiration ports on the catheter body in order to contain the infusate in the region surrounding the plaque or clot to be treated. In such cases, methods typically further comprise perfusing blood past the inflated balloons through a perfusion lumen in the catheter. Optionally, the methods may comprise varying the pumping rate of the lytic agent to provide a pulsed flow of lytic agent into the blood vessel.

### BRIEF DESCRIPTION OF THE DRAWINGS

In order to better understand the invention and to see how it may be carried out in practice, some preferred embodiments are next described, by way of non-limiting examples only, with reference to the accompanying drawings, in which like reference characters denote corresponding features consistently throughout similar embodiments in the attached drawings.

FIG. 1A exemplarily illustrates a proximal section of an atherolysis catheter apparatus comprising multiple ports.

FIG. 1B exemplarily illustrates a perspective view of a flexible distal section of the atherolysis catheter apparatus, showing multiple openings.

FIG. 2 exemplarily illustrates an enlarged view of a distal end of the flexible distal section of the atherolysis catheter apparatus, showing a distal perfusion opening.

FIG. 3 exemplarily illustrates an orthogonal view of the flexible distal section 103 of the atherolysis catheter apparatus, showing one of the proximal perfusion openings proximal to the proximal isolatation balloon on the flexible distal section.

FIG. 4 exemplarily illustrates a perspective view of the flexible distal section of the atherolysis catheter apparatus, showing balloon openings, the distal perfusion opening, and multiple proximal perfusion openings on the flexible distal section.

FIG. 5 exemplarily illustrates a cross sectional view of the flexible distal section of the atherolysis catheter apparatus, showing multiple lumens disposed in a space defined within the flexible distal section.

FIGS. 6A-6D exemplarily illustrate cross sectional views of the flexible distal section of the atherolysis catheter apparatus, showing different profiles of the atherolysis catheter apparatus.

FIGS. 7A-7B illustrate tables showing example dimensions of the different profiles of the atherolysis catheter apparatus and their corresponding characteristics.

FIG. 8 exemplarily illustrates a method for dissolving and/or modifying plaque in a vascular region of a patient using the atherolysis catheter apparatus.

FIG. 9 illustrates a distal portion of a catheter body with a portion broken away to show the infusion lumen and the aspiration lumen. A pump and pressure-responsive valves are shown in the infusion lumen.

FIGS. 10A-10C illustrate operation of an exemplary pressure-responsive valve in accordance with the principles of the present invention.

FIGS. 11A and 11B illustrate structure for attaching a motor to a pump of the type shown in FIG. 9.

### DETAILED DESCRIPTION OF THE INVENTION

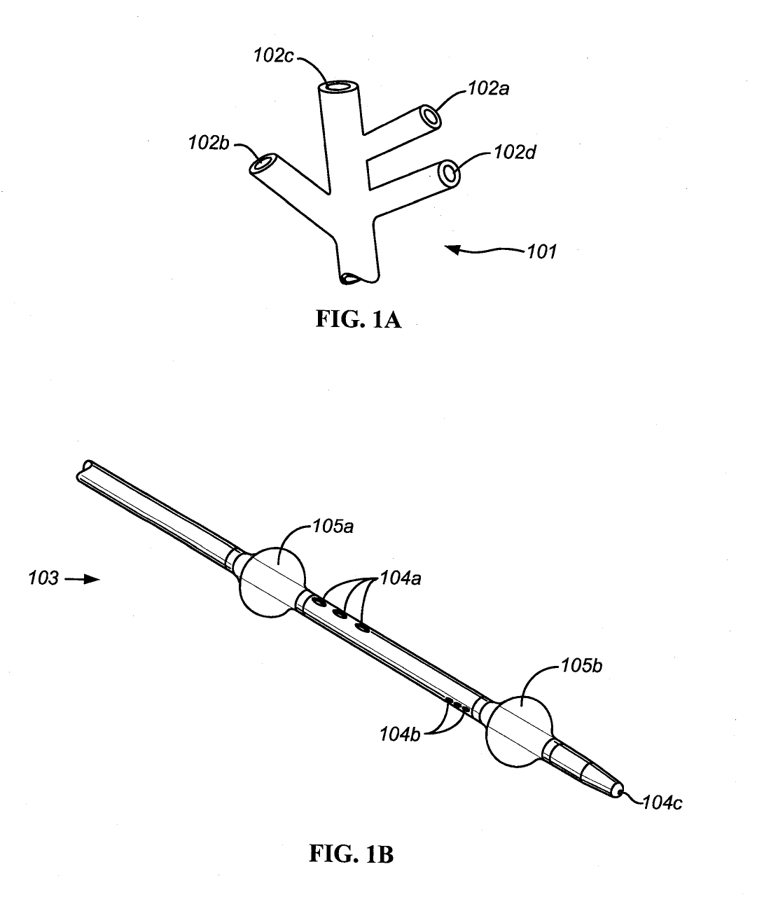

Disclosed herein is an atherolysis catheter apparatus comprising a proximal section 101, a flexible distal section 103, and one or more isolation balloons 105 as exemplarily illustrated in FIGS. 1A-1B. The atherolysis catheter apparatus disclosed herein is, for example, an over-the-wire catheter or a monorail catheter. FIG. 1A exemplarily illustrates the proximal section 101 (typically a hub) of the atherolysis catheter apparatus. The proximal section 101 comprises multiple ports 102a-102d that are in fluid communication with one or more lumens 501a-501f, as exemplarily illustrated in FIG. 5, of the flexible distal section 103 of the atherolysis catheter apparatus for delivering and collecting fluids, for example, plaque dissolution fluids, and other interventional elements, for example, a guide wire, a filter wire, etc., to and from the flexible distal section 103. As used herein, the term “fluid communication” refers to ability to exchanges fluids, for example, liquids and gases, to and from the cavity or blood vessel of a patient's anatomy. Also, as used herein, the term “cavity” refers to lumen of vascular regions, for example, blood vessels such as human arteries, extremities such as, peripheral arteries of the patient's legs, a carotid artery, a renal artery of the patient's kidney, etc., where plaque is formed. The vascular region referred to herein is, for example, an artery of the patient's heart. The proximal section 101 of the atherolysis catheter apparatus stays outside the patient's body.

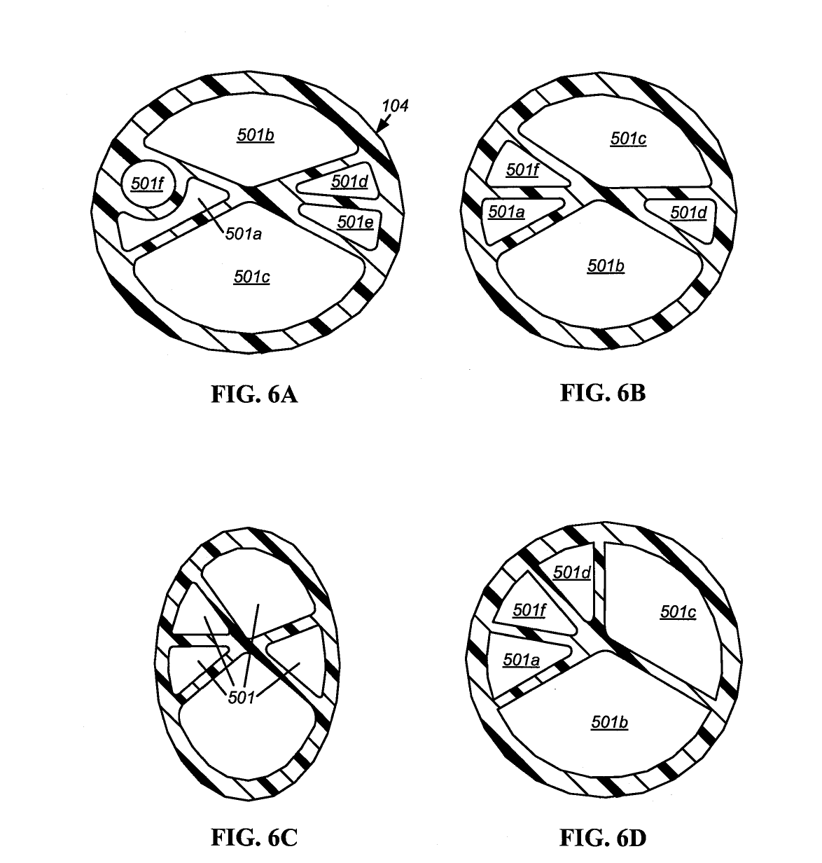

The ports 102a-102d of the proximal section 101 are connected through multiple lumens 501a-501f to one or more openings 104a and 104b in the flexible distal section 103 of the atherolysis catheter apparatus. For example, an infusion lumen 501a, as disclosed in the detailed description of FIG. 6A, is in fluid communication with, for example, a port 102a of the proximal section 101 for delivering a plaque dissolution fluid to a blood vessel, for example, an artery of a patient; an aspiration lumen 501b is in fluid communication with, for example, a port 102b of the proximal section 101 for collecting the delivered plaque dissolution fluid with dissolved plaques and plaque parts from the patient's artery; and balloon lumens 501d-501e are in fluid communication with the port 102d respectively for inflating and deflating the isolation balloons 105a and 105b. Furthermore, a guide wire lumen 501f is in fluid communication with, for example, a port 102c of the proximal section 101. A guide wire or filter wire that extends from the port 102c of the proximal section 101 outside the patient's body to the distal end of the flexible distal section 103. The guide wire or filter wire is inserted through the port 102c of the proximal section 101 and passes through the guide wire lumen 501f in the flexible distal section 103 through a wire opening 104c.

FIG. 1B exemplarily illustrates a perspective view of the flexible distal section 103 of the atherolysis catheter apparatus, showing multiple openings 104a and 104b. The flexible distal section 103 of the atherolysis catheter apparatus extends from and is connected to the proximal section 101 of the atherolysis catheter apparatus via one or more lumens 501a-501f enclosed within the flexible distal section 103. The flexible distal section 103 is a tubular structure comprising the lumens 501a-501f and one or more openings 104a and 104b. The lumens 501a-501f are disposed in a space defined within the flexible distal section 103. One or more of the lumens 501a-501f are in fluid communication with one or more ports 102a-102d of the proximal section 101. The lumens 501a-501f transport fluids and interventional elements to and from a cavity of the patient's blood vessel, for example, an artery. The atherolysis catheter apparatus is inserted into the patient's artery such that there is a space left between the atherolysis catheter apparatus and the inner surface of the artery for delivering the fluids.

The openings 104a and 104b are configured at predetermined positions on the flexible distal section 103 of the atherolysis catheter apparatus. The openings 104a and 104b are in fluid communication with one or more lumens 501a-501f in the flexible distal section 103 for delivering and collecting the fluids, plaque, etc., and for enabling passage of interventional elements to and from the cavity of the patient's blood vessel.

One or more isolation balloons 105a and 105b are disposed at predetermined positions on the flexible distal section 103. The isolation balloons 105a and 105b are in fluid communication with one or more of the openings 104a and 104b on the flexible distal section 103 for enabling selective inflation and deflation of the isolation balloons 105a and 105b. In an embodiment, a distal isolation balloon 105b is positioned near the distal end of the flexible distal section 103 of the atherolysis catheter apparatus and a proximal isolation balloon 105a is positioned away from the distal end of the atherolysis catheter apparatus. The isolation balloons 105a and 105b are inflated to create a space for delivering the fluids to the cavity or the blood vessel of the patient's anatomy, for widening a narrowed blood vessel, and for reducing spillage of the fluids and the plaque to the rest of circulation of the patient's anatomy. The isolation balloons 105a and 105b create an isolated space where the plaque dissolving fluid is delivered to dissolve the plaque. Moreover, during the time of delivering the plaque dissolving fluid, the isolation balloons 105a and 105b reduce spilling over of the plaque dissolving fluid and dissolved plaque to the rest of the circulation. The distance between the isolation balloons 105a and 105b is variable depending on size of atherosclerotic area that requires treatment. For example, the distance between the proximal isolation balloon 105a and the distal isolation balloon 105b is about 40 mm. In another embodiment, the distance between the isolation balloons 105a and 105b is variable depending on size of atherosclerotic area that needs to be treated.

In an embodiment, one or more of the isolation balloons 105a and 105b are used to perform angioplasty to certain atherosclerotic areas. The isolation balloons are filled with a fluid, for example, a liquid, a gas, etc. In an example, the isolation balloons are filled with a fluid, for example, by delivering an inflation medium through one of the ports 102a-102d of the proximal section 101 of the atherolysis catheter apparatus located outside the patient's body. In an embodiment, the isolation balloons 105a and 105b are connected to separate lumens 501d and 501e and port 102d. In another embodiment, the isolation balloons 105a and 105b share a lumen 501d or 501f and a port 102d.

The openings 104a and 104b configured on the flexible distal section 103 of the atherolysis catheter apparatus are, for example, one or more infusion openings 104b, one or more aspiration openings 104a, a guide wire opening 104c, one or more balloon openings 104d, one or more distal perfusion openings 104b, and one or more proximal perfusion openings 104a. The configurations of the openings 104a and 104d on the flexible distal section 103 of the atherolysis catheter apparatus are interchangeable and can be arranged in multiple different configurations.

The infusion openings 104b are configured at predetermined positions on the flexible distal section 103. One or more infusion openings 104b are in fluid communication with one or more of the ports 102a-102d of the proximal section 101 through one or more of the lumens 501a-501f in the flexible distal section 103, delivers fluids to the patient's blood vessel, where the fluids are injected through one or more of the ports 102a-102d outside the patient's body. The positioning of the infusion openings 104b on the flexible distal section 103 can be varied for different atherolysis catheter apparatuses. For example, one or more infusion openings 104b are positioned between the proximal isolation balloon 105a and the distal isolation balloon 105b, but closer to the distal isolation balloon 105b as exemplarily illustrated in FIG. 1B. In another example, the infusion openings 104b are centrally positioned between the proximal isolation balloon 105a and the distal isolation balloon 105b. In another example, the infusion openings 104b are positioned between the proximal isolation balloon 105a and the distal isolation balloon 105b, but closer to the proximal isolation balloon 105a.

The aspiration openings 104a are configured at predetermined positions on the flexible distal section 103. One or more aspiration openings 104a, in fluid communication with one or more of the ports 102a-102d of the proximal section 101 through one or more of the lumens 501a-501f, collects fluids, for example, solvents, dissolved plaque, small pieces of the plaque, etc., from the patient's blood vessel. The positioning of the aspiration openings 104a on the flexible distal section 103 can be varied for different atherolysis catheter apparatuses. For example, the aspiration openings 104a are positioned between the proximal isolation balloon 105a and the distal isolation balloon 105b, but closer to the proximal isolation balloon 105a as exemplarily illustrated in FIG. 1B. In another example, the aspiration openings 104a are centrally positioned between the proximal isolation balloon 105a and the distal isolation balloon 105b. In another example, the aspiration openings 104a are positioned between the proximal isolation balloon 105a and the distal isolation balloon 105b, but closer to the distal isolation balloon 105b.

In an embodiment, a radiological marker, for example, radiopaques are localized at either the inner side or on the outer side of each of the proximal isolation balloon 105a and the distal isolation balloon 105b. The radiological marker is a substance that does not allow radiation, for example X-rays, to penetrate through the radiological marker and hence enhances the X-ray pictures of the atherolysis catheter apparatus and enhances their visibility.

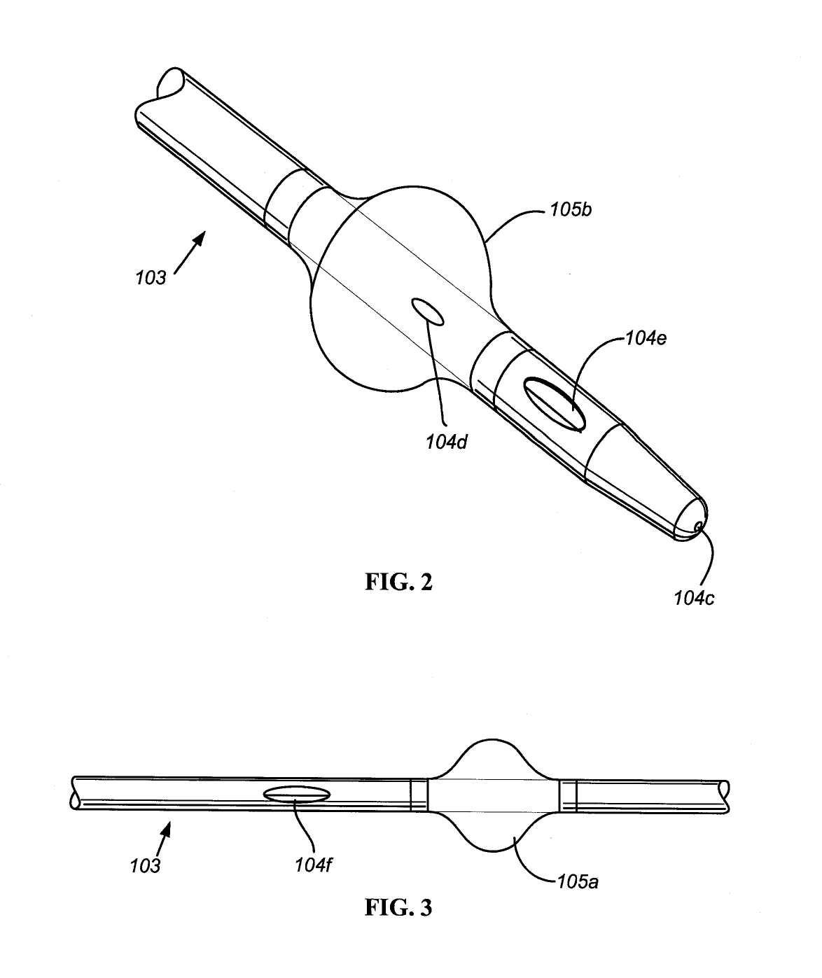

FIG. 2 exemplarily illustrates an enlarged view of the distal end of the flexible distal section 103 of the atherolysis catheter apparatus, showing a distal perfusion opening 104b. The distal end of the atherolysis catheter apparatus has the guide wire opening 104e, which represents the tip of the guide wire lumen 501f for passing the guide wire or the filter wire. The distal end of the flexible distal section 103 tapers to an atraumatic tip profile.

The guide wire or the filter wire passes through one of the ports 102 of the proximal section 101 into the cavity of the patient's anatomy, blood vessel, or artery via the guide wire lumen 501f that extends from the port 102 of the proximal section 101 to the distal end of the flexible distal section 103 of the atherolysis catheter apparatus. In the atherolysis catheter apparatus disclosed herein, the filter wire is utilized for preventing embolization of plaque pieces in the cavity of the patient's anatomy or blood vessel or artery. The guide wire or the filter wire is deployed into the patient's anatomy before advancing the atherolysis catheter apparatus into the cavity of the patient's anatomy and is retrieved at the end of the procedure. The guide wire crosses the area of intended plaque dissolution. If the filter wire is used then the filter wire is deployed distal to the area of intended plaque dissolution. The atherolysis catheter apparatus is then advanced over the back end of the guide wire or the filter wire through the guide wire lumen 501f at the distal end of the flexible distal section 103 of the atherolysis catheter apparatus. At the end of the plaque dissolving session, the atherolysis catheter apparatus is first removed, and then the guide wire or the filter wire is retrieved.

The distal perfusion openings 104b are is configured on the distal end of the flexible distal section 103 of the atherolysis catheter apparatus. The distal perfusion openings 104a are in fluid communication with the proximal perfusion openings 104a through a lumen 501c in the flexible distal section 103, allows blood flow to the artery when the isolation balloons 105a and 105b are inflated.

FIG. 3 exemplarily illustrates an orthogonal view of the flexible distal section 103 of the atherolysis catheter apparatus, showing one of the proximal perfusion openings 104f proximal to the proximal isolation balloon 105a on the flexible distal section 103. One or more distal perfusion openings 104c are configured at predetermined positions on the flexible distal section 103. The positioning of the perfusion openings 104e and 104f on the flexible distal section 103 can be varied for different atherolysis catheter apparatuses. For example, a proximal perfusion opening 104f is positioned proximal to the proximal isolation balloon 105a and connects to a distal perfusion opening 104 positioned distal to the distal isolation balloon 105b through one or more perfusion lumens 501c within the flexible distal section 103. The perfusion openings 104c and 104f connected by the perfusion lumens 501c allow blood flow to the artery when the isolation balloons 105a and 105b are inflated.

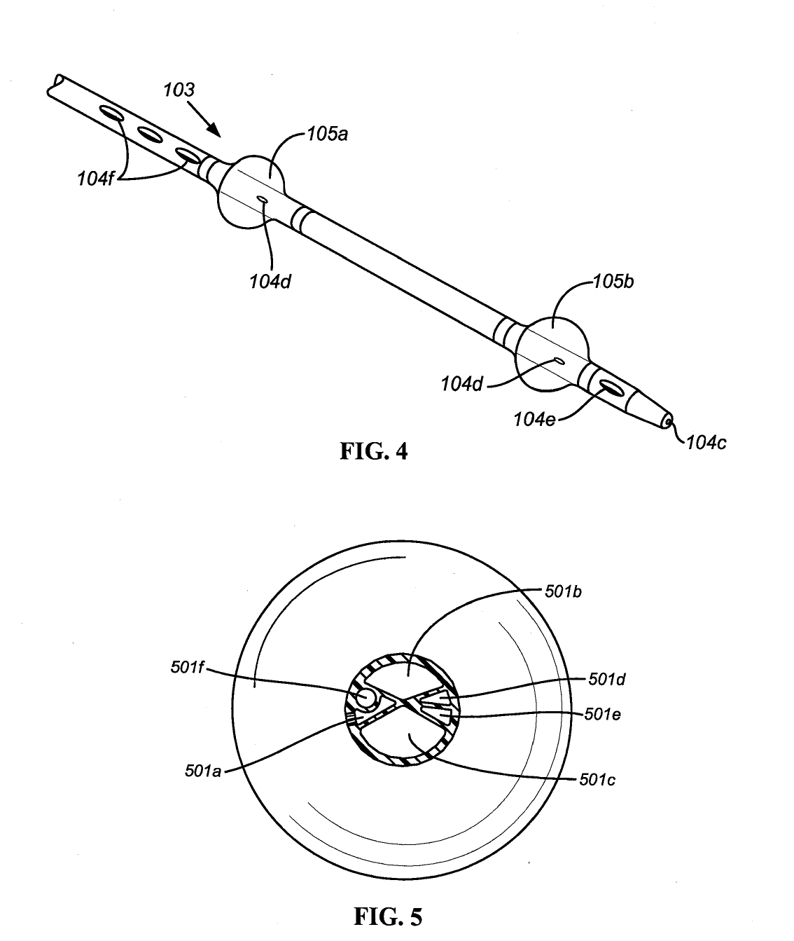

FIG. 4 exemplarily illustrates a perspective view of the flexible distal section 103 of the atherolysis catheter apparatus, showing balloon openings 104d, the distal perfusion opening 104e, and multiple proximal perfusion openings 104f on the flexible distal section 103. The balloon openings 104d are configured at predetermined positions on the flexible distal section 103 based on positioning of the isolation balloons 105a and 105b. The balloon openings 104d positioned inside the flexible distal section 103 covered by the isolation balloons selectively inflate and deflate the isolation balloons. The balloon openings 104d are in fluid communication with one or more ports 102d in the proximal section 101 of the atherolysis catheter apparatus through one or more of the lumens 501d. In an embodiment, the balloon openings 104d are connected to separate lumens 501d and ports 102d. In another embodiment, the balloon openings 104d share a single lumen 501d and a port 102d.

FIG. 5 exemplarily illustrates a cross sectional view of the flexible distal section 103 of the atherolysis catheter apparatus, showing multiple lumens 501a-501f disposed in a space defined within the flexible distal section 103. The atherolysis catheter apparatus defines different lumens 501a-501f, for example, an infusion lumen 501a, an aspiration lumen 501b, a perfusion lumen 501c, balloon lumens 501d and 501e, and a guide wire lumen 501f.

The infusion lumen 501a has one or more infusion openings 104b on the flexible distal section 103 of the atherolysis catheter apparatus to deliver fluids, for example, plaque dissolution fluids, to the cavity of the patient's anatomy. The aspiration lumen 501b is in fluid communication with one or more aspiration openings 104a on the flexible distal section 103 of the atherolysis catheter apparatus between two isolation balloons 105a and 105b to collect fluids from the cavity of the patient's anatomy. The perfusion lumen 501c is in fluid communication with the perfusion openings 104e and 104f.

The isolation balloons 105a and 105b, for example, the proximal isolation balloon 105a and the distal isolation balloon 105b are in fluid communication with a single balloon lumen 501a or separate balloon lumens 501a and 501d via the balloon openings 104d. If the isolation balloons have separate lumens, the proximal isolation balloon 105a and the distal isolation balloon 105b may be inflated and deflated together or in different sequences. If the proximal isolation balloon 105a and the distal isolation balloon 105b share a single balloon lumen, the proximal isolation balloon 105a and the distal isolation balloon 105b are inflated and deflated at simultaneously. The balloon lumens are in fluid communication with one or more ports 102d in the proximal section 101 of the atherolysis catheter apparatus which is located outside the patient's body.

In order to achieve perfusion and prevent ischemia while the proximal isolation balloon 105a and the distal isolation balloon 105b are inflated, there are one or more additional openings 104f on the flexible distal section 103, proximal to the proximal isolation balloon 105a that are in fluid communication with, for example, a perfusion lumen 501c. The other end of the perfusion lumen 501c is in fluid communication with one or more additional distal perfusion openings 104e on the flexible distal section 103, distal to the distal isolation balloon 105b. In an embodiment, some of the lumens 501a-501f are combined to achieve dual functions. For example, perfusion can be achieved through the same guide wire lumen 501f.

In an embodiment, a guide wire or a filter wire passes through a guide wire lumen 501f extending from a port 102c of the proximal section 101 outside the patient's body to the distal end, that is, the tip of the flexible distal section 103 of the atherolysis catheter apparatus. The atherolysis catheter apparatus advances over the guide wire or filter wire that passes through the guide wire lumen 501f. In another embodiment, the guide wire lumen 501f performs perfusion by incorporating one or more additional openings proximal to the proximal isolation balloon 105a and distal to the distal isolation balloon 105b respectively. The size of the guide wire is variable and depends on the size of the blood vessel to be treated. In an example, the guide wire is about 0.014 inches in diameter. The diameter of the guide wire can be configured depending on the size of the blood vessel to be treated.

FIGS. 6A-6D exemplarily illustrate cross sectional views of different embodiments of the flexible distal section 103 of the atherolysis catheter apparatus, showing different profiles of the atherolysis catheter apparatus. The lumen numbering conforms to that for previously embodiments with the generic reference number 501 in FIG. 6C indicating an embodiment where the lumens are interchangeable. The diameter of the flexible distal section 103 of the atherolysis catheter apparatus is sized by the French catheter scale (F). An atherolysis catheter apparatus with a diameter of size 8 F is exemplarily illustrated in FIG. 6A. An atherolysis catheter apparatus with a diameter of size 7 F is exemplarily illustrated in FIG. 6B. An atherolysis catheter apparatus with a diameter of size 7 F is exemplarily illustrated in FIG. 6C. An atherolysis catheter apparatus with a diameter of size 7 F or 8 F is exemplarily illustrated in FIG. 6D. These catheter diameters can range from about 2.8 F to about 40 F depending on the size of the blood vessels to be treated.

Consider an example where the atherolysis catheter apparatus defines a dedicated infusion lumen 501a in fluid communication with one or more openings 104a and 104b on the flexible distal section 103 of the atherolysis catheter apparatus. The dedicated infusion lumen 501a delivers the fluids from one of the ports 102a-102d of the proximal section 101 to the cavity of the patient's anatomy or blood vessel like artery via, for example, one or more infusion openings 104b. The aspiration lumen 501b then collects the fluids with dissolved plaque and small pieces of plaque from the cavity of the patient's anatomy or blood vessel and to one of the ports of the proximal section 101 via another opening, for example, the aspiration opening 104a. In an example, the aspiration lumen 501b collects the fluids from the cavity of the patient's anatomy by use of suction.

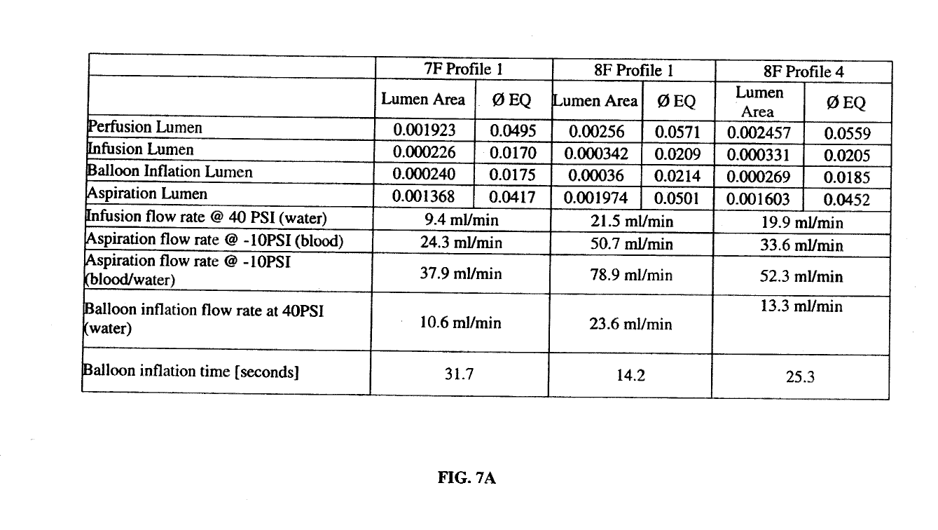

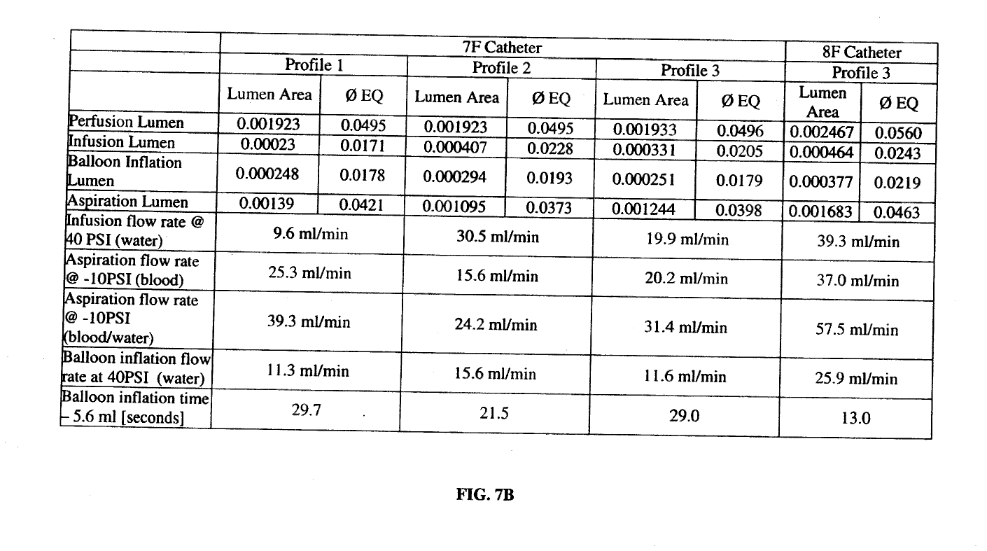

FIGS. 7A-7B illustrate tables showing the example dimensions of the different profiles of the atherolysis catheter apparatus and their corresponding characteristics. FIG. 7A shows the infusion flow rate of water at 40 pounds per square inch (PSI), the aspiration flow rate of blood at −10 PSI, the aspiration flow rate of blood and water at −10 PSI, the balloon inflation flow rate of water at 40 PSI, and the balloon inflation time for the lumen areas and equivalent diameter (EQ) for 7 F profile 1, 8 F profile 1, and 8 F profile 4 atherolysis catheter apparatuses. FIG. 7B shows the infusion flow rate of water at 40 PSI, the aspiration flow rate of blood at −10 PSI, the aspiration flow rate of blood and water at −10 PSI, the balloon inflation flow rate of water at 40 PSI, and the balloon inflation time for different lumen areas and EQ for 7 F profile 1, 7 F profile 2, 7 F profile 3, and 8 F profile 3 atherolysis catheter apparatuses.

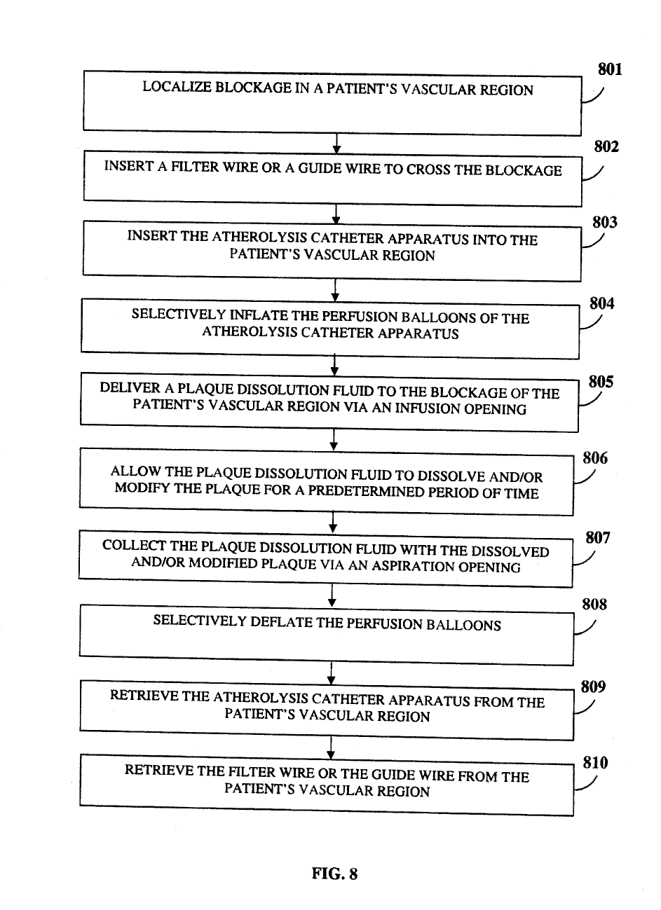

FIG. 8 exemplarily illustrates a method for dissolving and/or modifying plaque in a vascular region of a patient using the atherolysis catheter apparatus disclosed herein. The plaque, for example, composed of lipids, calcium, and connective tissue builds up in the vascular region leading to blockages in the vascular region. The blockages in the vascular region are localized 801 by performing, for example, diagnostic angiography. The guide wire or the filter wire is then inserted 802 into the vascular region to cross the blockages in the vascular region, using interventional equipment, for example, guide catheters, sheaths, torque devices, etc., under X-ray fluoroscopy guidance. The atherolysis catheter apparatus disclosed herein is inserted 803 into the vascular region of the patient by advancing the guide wire lumen 501f over the back end of the guide wire or the filter wire. The atherolysis catheter apparatus is positioned such that, the atherosclerotic area to be treated is located between the proximal isolation balloon 105a and the distal isolation balloon 105b. The isolation balloons 105 are selectively inflated 804 by introducing air or another fluid through one or more of the ports 102a-102d of the proximal section 101. One or more lumens 501a-501f, for example, the infusion lumen 501a receives a plaque dissolution fluid or solvent via one of the ports 102a-102d of the proximal section 101 and delivers 805 the plaque dissolution fluid to the blockage of the vascular region of the patient via the infusion opening or openings 104b. The plaque dissolution fluid is allowed 806 to dissolve and/or modify the plaque present in the blockage of the vascular region of the patient for a predetermined period of time.

Another one of the lumens 501a-501f, for example, the aspiration lumen 501b collects 807 the delivered plaque dissolution fluid with the dissolved and/or modified plaque from the vascular region of the patient via the aspiration opening or openings 104a after the predetermined period of time. When the delivered plaque dissolution fluid and the dissolved and/or modified plaque from the vascular region of the patient is collected, the isolation balloons 105 are selectively deflated 808 by suctioning the air or other fluid from the isolation balloons 105 via the balloon openings 104d. The atherolysis catheter apparatus disclosed herein are thereafter retrieved 809 from the patient's vascular region. The guide wire or the filter wire is then retrieved 810 from the artery after the dissolution of the plaque. The method disclosed herein can be repeated as needed. The method and atherolysis catheter apparatus disclosed herein can be used alone or in conjugation with other treatment modalities, for example, balloon angioplasty, atherotomy, stenting, etc.

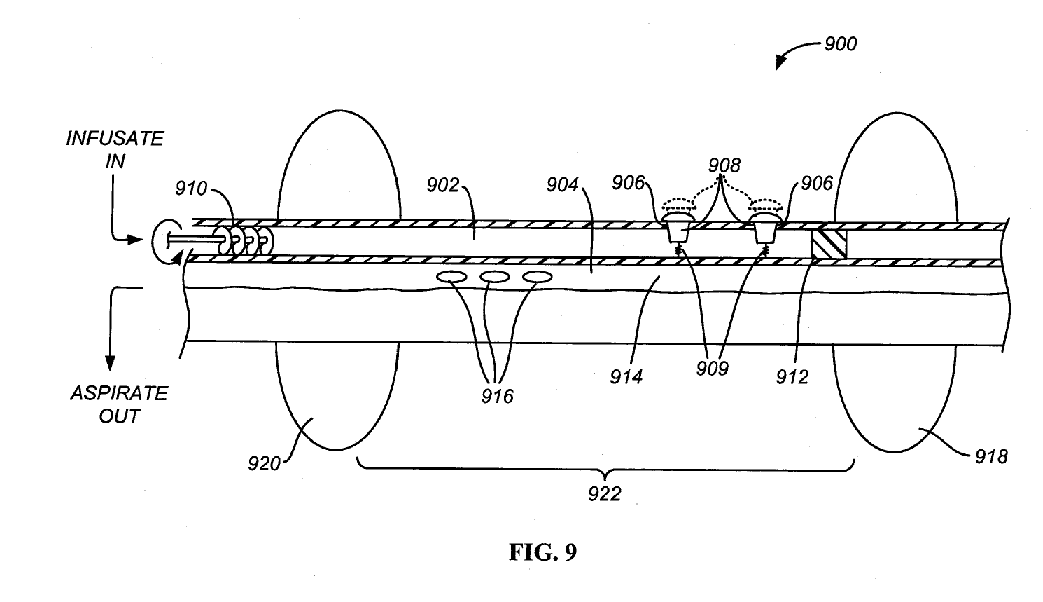

Referring now to FIG. 9, incorporation of a pump and one or more pressure-responsive valves into the infusion lumen of an atherolysis catheter according to the present invention is illustrated. The overall catheter construction, including the inclusion of axially spaced-apart isolation balloons was well described above, and the following discussions will provide more detail on how to incorporate the pressure-responsive valves. In a distal portion 900 of the atherolysis catheter, an infusion lumen 902 and aspiration lumen 904 may be formed as described previously. At least one valved infusion port 906 is formed in a wall of the infusion lumen 904 so that infusate passing through the lumen may pass outwardly to a region surrounding the catheter for treatment. Each valve infusion port 906 may include a valve plug 908 which is resiliently mounted in order to open in response to a positive pressure within the infusion and to close when said pressure is lowered. Conveniently, the pressure-responsive valve may comprise a spring element 909, optionally a coil spring but alternatively any type of tension spring, which is mounted to draw the associated plug 908 downward to close against a valve seal defined by the associated infusion port 906. In order to increase the pressure of infusate flowing in through the infusion lumen 902, a rotary pump 910, such as a screw pump, turbine pump, or the like, is provided in the infusion lumen, preferably within a short distance from the valve structures, typically within 1 cm to 40 cm, usually within 1 cm to 10 cm. The pumping element 910 is mounted to rotate in order to raise the pressure and flow rate of infusate entering the infusion lumen through a proximate port on the catheter (not shown), typically which is part of the proximal catheter hub structure.

The distal portion 900 of the catheter preferably includes a distal isolation balloon 918 and a proximal isolation balloon 920 which are spaced-apart on either side of the infusion ports 916 and the aspiration ports 916 which open into aspiration lumen 914. The catheter structure illustrated in FIG. 9 will typically also include one or more perfusion lumens, guide wire lumens, balloon inflation lumens, and the like, each of which was well described in connection with previous embodiments of the present invention.

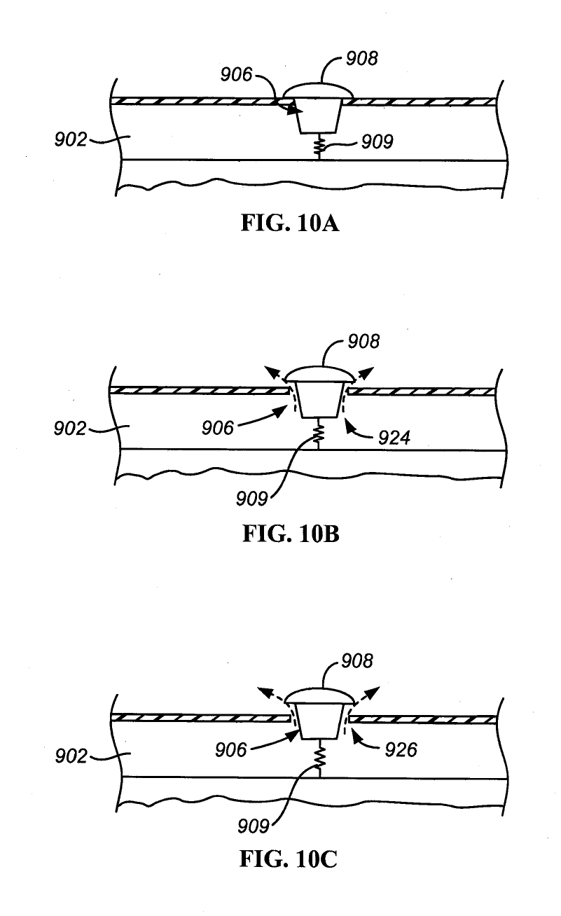

Referring now to FIGS. 10A through 10C, an assembly of the infusion port 906, valve plug 908, and spring 909 will be described in more detail. When the pressure in the aspiration lumen 914 (FIG. 9) is below the threshold pressure level, as described above, the spring 909 will maintain sufficient downward or closing force on the valve plug 908 so that the valve plug is seated within the infusion port 906, as shown in FIG. 10A. When the infusion pressure reaches a level above the threshold pressure, as shown in FIG. 10B, the valve 908 will begin to rise from the seat of infusion port 906, thus creating an annular orifice or flow path 924 which allows a first rate of infusate flow from the valve into the treatment region 922 (FIG. 9). As the infusion pressure rises, optionally by controlling the speed of pump 910, the valve plug 908 will rise further, enlarging the annular orifice 926 and allowing a greater flow rate of infusate into the treatment region 922, as shown in FIG. 10C. It will be appreciated that control of the opening pressures and resulting flow rates can be adjusted by choosing the spring constant of spring 909.

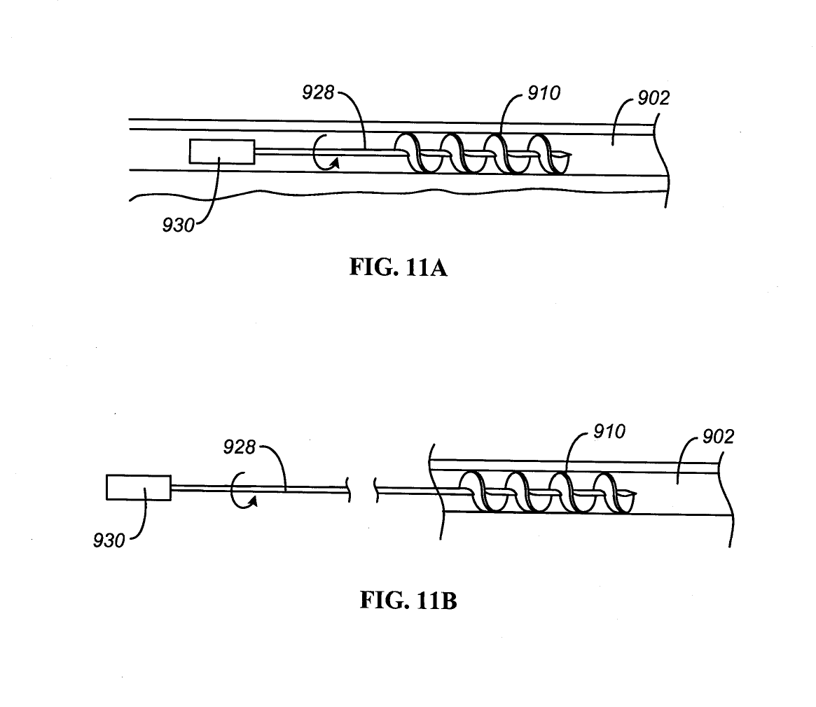

Referring now to FIGS. 11A and 11B, the screw or the pump mechanism 910 may be driven by a rod-like drive shaft 928 which is attached to a drive motor 930 which is mounted externally of the catheter. The motor 930 may also be disposed in the infusion lumen 902, as illustrated in FIG. 11A, or alternatively may be disposed outside of the infusion lumen, typically an approximal hub of the catheter, where the drive shaft 928 extends through the major length of the catheter lumen. The source of power may be placed next to the motor in the infusion port or outside of the catheter with electric wire that extends through the major length of the catheter lumen.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may make numerous modifications thereto and changes may be made without departing from the scope and spirit of the invention in its aspects.

What is claimed is:

1. An atherolysis catheter comprising:
a catheter body comprising an infusion lumen having at least one infusion port at a distal end thereof and an aspiration lumen having at least one aspiration port at a distal end thereof;
a pump disposed in the infusion lumen and adapted to flow an infusate through the infusion lumen toward the infusion port; and
a pressure-responsive valve disposed at the infusion port, wherein the valve is normally closed but opens in response to flow of the infusate caused by the pump.

2. An atherolysis catheter as in claim 1, wherein the pressure-responsive valve comprises a valve plug reciprocatably mounted in the infusion port, wherein the infusion port defines a valve seat.

3. An atherolysis catheter as in claim 2, wherein the valve plug is attached to a spring which acts to close the plug against infusate pressure.

4. An atherolysis catheter as in claim 3, wherein the valve plug has a conical taper which centers in the infusion port when the valve is closed and which opens to provide an annular gap which acts as a nozzle jet to distribute the infusate in a blood vessel being treated.

5. An atherolysis catheter as in claim 1, further comprising a pair of axially spaced-apart isolation balloons, with a distal balloon disposed on the catheter body distally of the infusion port and the aspiration port and a proximal balloon disposed proximally of the infusion port and the aspiration port.

6. An atherolysis catheter as in claim 5, wherein the catheter body has a perfusion lumen having an inlet port on one side of the pair of isolation balloons and an outlet port on another side of the pair of isolation balloons.

7. An atherolysis catheter as in claim 1, further comprising a motor coupled to the pump.

8. An atherolysis catheter as in claim 7, wherein the motor is disposed in the infusion lumen.

9. An atherolysis catheter as in claim 7, wherein the motor is disposed outside of the infusion lumen and connected to the pump by a drive cable disposed in the infusion lumen.

10. An atheterolysis catheter as in claim 7, wherein the motor is adapted to be driven variably to pulse the flow of infusate through the infusion lumen and out of the infusion port.

11. A method for treating vascular occlusions, said method comprising:
positioning a distal end of a catheter near an occlusion in a blood vessel;
pumping a lytic agent through an infusion lumen and out an infusion port on the catheter, wherein the lytic agent opens a pressure-responsive valve in the infusion port, wherein the valve sprays the lytic agent into the occlusion in the blood vessel; and
aspirating the lytic agent and lysed products from the blood vessel through an aspiration port and aspiration lumen in the catheter body.

12. A method as in claim 11, wherein a pumping rate of the lytic agent is varied to provide a pulsing flow rate of lytic agent into the blood vessel.

13. A method as in claim 11, wherein the in-line screw pump is rotated by a motor disposed in the infusion lumen.

14. A method as in claim 11, wherein the in-line screw pump is rotated by a drive cable disposed in the infusion lumen, said drive cable connected to a motor disposed outside of the infusion lumen.

15. A method as in claim 11, further comprising inflating a distal isolation balloon distally of the infusion and aspiration ports and a proximal isolation balloon proximally of the infusion and aspiration ports.

16. A method as in claim 11, further comprising providing a perfusion lumen in the catheter to allow blood bypass of the isolation balloons.

17. A method as in claim 11, wherein pumping comprises driving a pump disposed within the infusion lumen.

18. A method as in claim 11, wherein driving comprises rotating an in-line screw pump disposed in the infusion lumen.
